(19) United States
(12) Patent Application Publication
(10) Pub. No.: US 2010/0121312 A1
(43) Pub. Date: May 13, 2010

(54) Catheter Shaft

(75) Inventors: Gerhard Gielenz, Wetzikon (CH); Eugen Hofmann, Zuerich (CH)

Correspondence Address: BIOTECH BEACH LAW GROUP , PC, 5677 OBERLIN DRIVE, SUITE 204, SAN DIEGO, CA, 92121, US

(73) Assignee: BIOTRONIK VI PATENT AG, Baar (CH)

(21) Appl. No.: 12/613,469

(22) Filed: Nov. 5, 2009

(30) Foreign Application Priority Data
Nov. 7, 2008 (DE) 10 2008 043 541.4

(51) Int. Cl. A61M 25/00; B29C 53/58

(52) U.S. Cl. 604/524; 156/195; 156/425

(57) ABSTRACT

The invention relates to a method for the production of at least one longitudinal section of a catheter, wherein the method comprises the winding of film, and the film is provided in the form of strips, the film strip is wound in the manner of a screw such that the side edges of the film strip extending in the manner of a thread overlap the film strip, and further a bonded connection is created in the overlapping region such that the longitudinal section has a closed and at least fluid-tight surface. The invention further relates to a catheter produced according to the method, and a device for carrying out the method.

### CROSS REFERENCE TO RELATED APPLICATIONS

This invention claims benefit of priority to Germany patent application serial number DE 10 2008 043 541.4, filed on Nov. 7, 2008; the contents of which are herein incorporated by reference in their entirety.

### FIELD OF THE INVENTION

The present invention relates to a method for the production of a longitudinal section of a catheter, the catheter including said longitudinal section, and a device for carrying out the method of production.

### BACKGROUND OF THE INVENTION

The catheter according to the invention is suitable for probing and for draining or filling human or animal hollow organs, and for the inflation or deflation of a balloon attached to a catheter, such as in vascular intervention, particularly in PTCA or neurovascular applications.

Such catheters, having a long shaft section including a hollow interior cross-section, must be able to withstand high loads of tension, pressure and torsion with an elastic behavior. Furthermore, the same should have a high bursting strength, and an interior lateral surface having a low roughness for improving the sliding behavior of instruments inserted into the catheter shaft.

In this regard there is the discrepancy of the desired low outer diameter and the inherent low wall thickness as opposed to the required mechanical strength. Catheter shafts having a low outer diameter with a low wall thickness and the required mechanical properties can be provided only at an increased production expense and therefore at a high cost.

Known catheter constructions, such as illustrated in U.S. Pat. No. 6,824,553 B1, U.S. Pat. No. 6,143,013, and U.S. Pat. No. 4,425,919, comprise metal mesh for reinforcing the catheter shaft, which are at least positively fitted to the continuous tubes. In order to realize a smooth outer surface, said catheters further have a coating across the metal mesh, which is also embodied as a continuous tube. Although catheter shafts produced in this manner have advantageous properties, said catheter shafts are, however, dimensioned with regard to the outer diameters and wall thicknesses thereof such that the same may not be utilized for applications in very narrow vessels, such as in particular in neurovascular surgery. Furthermore, the method of production of such catheter shafts is overall very extensive, and therefore cost-intensive.

Another method for the production of catheters, or the shafts thereof, is the extrusion of at least one component o the catheter shaft in the shape of a tube. EP 0 650 740 A1 discloses, for example. that an outer layer in the shape of a tube is extruded across the inner layer of a catheter shaft that is already present in the shape of a tube. This means that the extruded layer is therefore present at a hollow cross-section, in which the layer that is also embodied in the shape of a tube is arranged beneath the same. Currently, wall thicknesses of 70-80 μm may be realized by means of extrusion methods, depending on the material used and the diameter of the catheter shaft desired. Therefore, low wall thicknesses currently cannot be produced, because in this case the annular gap between the nozzle and the mandrel of the extrusion device would be dimensioned so narrow during the extrusion process, and thus the mass pressure would be so high that a stretching ratio of the extruder would be created, which would result in a molecule orientation in the extruder direction that would be too strong.

An alternative to the adjustment of an annular gap measurement that is too low is a higher extruder speed such that the tube is retracted from the extruder die more rapidly. In this method a stretching ratio that is too high would also be present at too high of a speed. The pronounced molecule orientation in the extruder direction results in anisotropic mechanical properties of the extrudate; that is to say in the increase of mechanical strength in the longitudinal direction with simultaneous reduction of the bursting strength and torsion strength. In contrast ideal isotropic catheter shafts of the same dimension would have an increased bursting and torsion strength at a sufficient tension and burst strength.

The pronounced molecule orientation in the extruder direction has an adverse effect, particularly in extruded tubes, when the extruded tube is stressed in radial direction, such as by means of applying an increased interior pressure during the inflating of a balloon attached to the catheter shaft. Furthermore, a molecule orientation that is too pronounced has a negative effect on welding or coating processes, also with regard to aging, since hardly any more controllable longitudinal shrinkages of the shaft may occur in these cases.

For this reason the catheter illustrated in EP 0 650 740 A1 has a layer in the tube walls including a relatively low wall thickness, however, said layer must be connected to a second interior layer for the purpose of realizing the mechanical manufacturing qualities necessary such that a two-layer hollow profile of the catheter shaft is embodied overall. Said construction consisting of two layers mandatorily brings about a relatively large diameter such that the catheter shaft is not accessible for use particularly in narrow vessels.

It is further known to equip catheters, or the shaft of a catheter, with a wrapped coating. Among others, US 2005/0059957 A1 discloses in FIG. 17 a tube that is sliced in a spiral shape, which is coated with a film. That is to say that the slit(s) in the tube extending in spiral shape are covered by the film. FIG. 18a illustrates a similar embodiment, wherein the film covering the tube that is slit in spiral shape also has slots that are embodied in a manner complementary to the slots in the tube. The first stated embodiment of US 2005/0059957 A1 has the disadvantage that a relatively large diameter of the catheter shaft is produced by means of the two layers of the catheter shaft (first layer: slit tube, second layer: film), which makes the use of the catheter in very narrow vessels impossible, as mentioned above. The second of the stated embodiments has the further disadvantage that the catheter shaft comprises no closed surface such that any fluids incorporated into the same may radially exit the shaft before they are able to fill a balloon, which is, for example, attached to the catheter shaft end.

### SUMMARY OF THE INVENTION

The invention is therefore based on the object of providing a method and a device for the production of a catheter, and a catheter, which has a low wall thickness with a low outer diameter of the catheter shaft, and which combines a high bursting strength with a tension, pressure, and torsion strength while having a smooth interior surface and sufficient capability of being further processed.

Said problem is solved by means of the method according to the invention for the production of at least one longitudinal section of a catheter according to claim 1, and by means of the catheter produced utilizing the same according to claim 10, and by means of the device suitable for carrying out the method according to claim 15. Advantageous embodiments of the method are stated in claims 2 to 9, and advantageous embodiments of the catheter are stated in the sub-claims 11 to 15.

The invention provides a method for the production of at least one longitudinal section of a catheter, wherein the method comprises the winding of film, which has previously been provided as a web, and is wound according to the invention in a screw-type manner such that the side edges of the film strip extending in the manner of a thread overlap the film strip. Subsequently, a bonded connection is created in the overlapping area such that the longitudinal section has a closed and at least fluid-tight surface. For this purpose the longitudinal section of the catheter is in particular the shaft, or an area of the shaft of a catheter, embodied as a hollow cylinder of a certain length, on which a balloon for expanding tissue may optionally be attached, or also one or more operating elements. The attachment of the balloon to the catheter shaft can be realized by means of welding. This is to say that the catheter longitudinal section may exclusively consist of film strip windings, which are connected to each other such that they form a tight surface. Optionally, said film strip windings may be fitted with a thin coating in retrospect. However, for this purpose even a coated longitudinal section is constructed according to the invention such that the coating is so thin as compared to the tube wall realized by the windings that at least 70 to 90% of the mechanical strength values of the longitudinal section arc realized by the windings, or coiling of the film strip, which are connected to each other.

Therefore, the catheter longitudinal section according to the invention does not comprise any metal windings, or wire mesh, such as are known according to prior art. Thus, no additional process steps must be performed for the production of a catheter, except for the connection of further catheter elements, such as a balloon, or operating elements and/or reworking.

The screw-type winding of the film strip has the effect that the side edges of the film strip have an incline like a thread of a screw. Said side edges of the film strip present in the form of a thread are the edges of the web being present in the form of strips extending in the longitudinal extension direction of the web. For this purpose the film strip is wound such that a hollow object having a closed surface and a rounded cross-section is created, wherein the winding and the bonded connection of the film strip are tight such that a leakage of fluid contained in the longitudinal section can be prevented.

Preferably, the winding and the bonded connection are embodied in a gas-tight manner.

In order to obtain a defined hollow cross-section of the longitudinal section the film strip may be wound on a mandrel in an overlapping manner, which is removed from the longitudinal section after the production of the bonded connection.

As an alternative it may also be provided that the film strip or multiple film strips are wound on an extremely thin-walled tube, wherein the thickness of the tube wall is significantly thinner, than the film strip thickness. The tube wall thickness is preferably less than or equal to 50 μm. That is to say that in this embodiment the catheter shaft receives the desired mechanical properties by means of the film strip attached to or on the tube, wherein at least 70 to 90% of the mechanical strength values of the longitudinal section are also realized by means of windings or coiling of the film strip. The tube without film strip to be equipped with the film strip would not meet the requirements of a catheter shaft.

One advantage of said embodiment is that a tube remaining in the wound up catheter shaft is utilized instead of a mandrel to be removed from the catheter shaft after the winding process, which simultaneously serves for the radial sealing of the catheter shaft.

With the method according to the invention catheters shafts can be produced having extraordinarily small wall thicknesses, and therefore small outer diameters, which are suitable for use in PTCA, or for neurovascular applications. For this purpose the film strip is preferably made from a biocompatible polymer. A catheter, or a catheter shaft, may be produced by means of the winding technique, which has ring-like reinforcement areas that are arranged next to each other via the overlapping regions of the windings, which bring about a significant resistance to bursting with the increase of the interior pressure within the catheter shaft. Generally, the longitudinal section, or the catheter shaft, is produced only from film material, thus substantially reducing the wall thickness as compared to the solutions according to prior art. Due to the overlapping region extending across the entire length of the longitudinal section, which extends in the manner of a screw line, the catheter shaft is also reinforced in longitudinal direction such that increased tension and pressure strength values are obtained. The carrying out of the method according to the invention is uncomplicated, and therefore cost-effective.

Due to the low wall thickness, and the resulting relatively large inner diameter, a relatively large inflation or deflation cross-section is provided for the gas exchange of a balloon attached to the longitudinal section for expanding tissue.

This enables a quick inflation or deflation of a balloon such that the blood flow in the vessel is not interrupted for too long.

The relatively large inner diameter of the produced catheter shaft enables easy insertion of a guide wire of a manipulation tool.

Advantageously, the bonded connection of the film strip is produced by means of welding. For this purpose the entire width of the overlapping region of the film strip does not necessarily need to be welded, but in order to realize the fluid-tight surface it suffices that only partial areas of the width of the overlapping area are connected in a bonded manner, however, the bond must be carried out across the entire length of the overlapping area. The welded connection may be carried out, for example, by means of heat exposure from an external heat source. The welding of the film materials to one another is therefore carried out in a sintering process. The heat source may be an oven, in which the complete longitudinal section is accommodated. As an alternative, the heat source may be an infrared heat source, by means of which a punctiform heating of the overlapping region may be carried out.

Furthermore, the film strips may be welded to each other in the overlapping regions by means of laser beams.

The welding is advantageously carried out, if the same occurs at an increase of the pressure on the film strip to be connected. Such pressure increase may be realized, for example, via heat shrinkable tubing, which is peeled off from the same after the cooling of the longitudinal section to be produced.

For this purpose said heat shrinkable tubing is comprised of PTFE, or a similar material, and is embodied in a high-temperature resistant manner. As an alternative the pressure may also be applied onto the film strip in a pressurized heating furnace by means of N₂, or another inert gas.

As an alternative to welding the film strip the invention provides that the bonded connection of the film strip is realized by means of gluing. For this purpose the glue must be applied to the film strip before the winding of the same, that is to say in the areas of the film strip provided for the overlapping. Of course, the glue should also be comprised of a biocompatible material.

The method according to the invention can be carried out in a simple and cost-effective manner in that only one film strip is wound for producing the longitudinal section, wherein said film strip partially overlaps itself This means that the side edges of the film strip extending in the manner of a screw overlap with the same film strip. Therefore, aside from the overlapping regions, a catheter longitudinal section is provided that has substantially only one tube wall made from one layer. The advantage of such a longitudinal section is the cost-effective production, and the extraordinarily small wall thickness, which ensures a large interior lumen of the catheter shaft. Furthermore, such a catheter longitudinal section comprises an increased bending flexibility.

As an alternative to the production of a catheter wound from only one film strip the method is carried out such that at least two film strips are wound for producing the longitudinal section, wherein each of the film strips partially overlaps itself and/or at least one of the other film strips. For this purpose the invention may provide that, for example, two film strips are wound parallel to each other and extending at a mutual incline, and are welded or glued. As an alternative the two film strips may be wound in a crossing manner, and connected in a bonding manner. More than two film strips may also be utilized, e.g. two film strips, for example, are processed parallel to each other, and a third or multiple further film strips are wound in a crossing manner to the same, and are welded or glued.

The method may further be embodied such that the film strips are wound simultaneously and bonded to each other simultaneously. That is to say that initially all coiling or windings are created and subsequently all bonded connections are created simultaneously, such as in a sintering furnace.

As an alternative the film strips may be chronologically wound in successive order, and also chronologically bonded to each other in a successive order. For this purpose the invention may provide that just as with the coiling of only one film strip, a film strip overlaps itself, simultaneously overlapping an already wound further film band, and that the bonded connection is created in the overlapping regions. This means that preferably with the coiling of one strip onto a hollow cylinder of a further strip already created by means of coiling, the windings of the second strip are connected to the first already wound strip.

For this purpose the closed and fluid-tight surface of the longitudinal section does not necessarily need to be realized by the coiling and bonded connection of a first wound film strip, but can result from the coiling and welding or gluing of a second or multiple further film strips onto the first film strip.

A further embodiment of the method is achieved in that multiple film strips are overlapped before coiling such that the same have a parallel course. For this purpose said overlapping film strips may already be bonded to each other before coiling, and welded or glued to each other in the overlapping region after coiling, that is to say at the windings, or optionally at the windings of further already wound film strips. In this manner, for example, a six-layer tube wall of the longitudinal section can be created by means of a double coiling of layers including a total of three film strips. The advantage of the catheter longitudinal sections being produced by means of multiple film strips in the increasing bursting and tension, as well as pressure strength in a wall thickness of the longitudinal section that is only slightly increased. For this purpose the coiling and also the bonded connection of the film strips may be carried out in one process step such that the production of a longitudinal section wound from multiple film strips also remains relatively cost-effective.

The method can further be embodied in an advantageous manner in that the edges of the overlapping regions are bonded to the film strip at the interior of the longitudinal section. As mentioned above, the bonded connection suffices for realizing a tight surface of the longitudinal section, if the same is carried out on at least one position of the width of the overlapping region. However, in order to ensure a smooth interior surface of the interior wall of the catheter section for improved intrinsic gliding properties, the edges of the overlapping regions are incorporated into the bonding process.

In contrast it is advantageous, if the edges of the overlapping regions are at least partially not incorporated into the bonded connection to the film strip at the exterior walls of the longitudinal section to be produced. Due to the fact that the edges are not welded or glued to the film strip positioned underneath, no stages are created that bring about an improved grip of the longitudinal section. Preferably, said embodiment should be utilized for areas of the longitudinal section, which are to serve as a grip or actuation end.

A particularly advantageous embodiment of the method provides that the film strip is produced by means of extrusion, and the side edges of the film strips provided extending in longitudinal direction extend in the extrusion direction. In an extruded film material the molecules are aligned in extrusion direction. Due to the fact that the side edges of the film strip extending in longitudinal direction are also aligned in extrusion direction, the molecule orientation is therefore also present in the direction of the longitudinal direction of the side edges of the film strip. This means that the molecule orientation approximately follows the bending course or the circumferential direction of the hollow profile cross-section of the catheter longitudinal shaft by means of the coiling of the film strip. This brings about a substantially greater capacity by an interior pressure present in the interior of the catheter shaft, which, for example, serves for filling a balloon that is attached to the catheter. For this purpose the film strips may be produced such that the film of a greater width can be extruded, which is subsequently cut apart such that the longitudinal edges of the strips produced extend in the extrusion direction.

As an alternative the invention may provide that film strips are extruded, which are not cut on the sides, but are wound according to the invention immediately after extrusion, optionally as an endless material. For this purpose the cooling of the film strip material heated for extrusion purposes can be advantageously utilized for a shrinking process of the film strip on a winding mandrel.

In order to produce a hollow cross-section of the catheter longitudinal section, having defined interior measurements, the film strip is wound onto a winding mandrel. Such a winding mandrel may be, for example, a wire mandrel or a solid material. The mandrel may be coated with separating means before the winding with film strips, such as Teflon or silicone oil in order to prevent a welding or gluing of the same to the film strip. The mandrel may further have a conical shape in order to create a conical cross-section of the longitudinal section. In this manner catheter longitudinal sections having a cylindrical shape can be produced, but also longitudinal sections having a hollow truncated shape. A particular embodiment of the method provides that the winding mandrel is comprised of a material having a melting point of below 80° C., and that such mandrel is melted out of the interior hollow space of the catheter longitudinal section to be produced in the bonded connection of the film strip windings and the related temperature increase. In this manner easy removal of the mandrel from the longitudinal section is possible, which must occur simultaneously with the welding of the film strip windings under the influence of heat. The time and cost expense for the production method is therefore reduced.

Furthermore, the method according to the invention is embodied in an advantageous manner, if the film strip is wound under tension force. Due to the elastic behavior of the film strip an improved sealing is obtained at the overlapping windings of the film strip.

In deviation from the production method, wherein the film strip is wound onto a winding mandrel, the method according to the invention may also be carried out such that the film strip is initially present as a strip spiral, and is subsequently pulled apart along the center axis of the spiral such that windings are created that overlap each other, the overlapping regions of which are subsequently bonded to each other. A longitudinal section having windings can be produced in a simple manner using this method, without having to guide the film strip across a longer path in windings during the production process. Furthermore, an at least slightly conical shape of the catheter shaft may be created in a simple manner by means of said production method.

A longitudinal section of the catheter or of the catheter shaft may be produced by means of the method according to the invention. For this purpose, however, it should not be ruled out that after the winding and the bonded connection of the film strip windings further processing steps may occur, such as coating, surface treatment, shaping processes, heat treatment, or radiation for modifying the properties of the longitudinal section. This means that, for example, after the bonded connection, a coating process may be carried out such that the longitudinal section is comprised not only of the winding layer, but also of additional layers. For this purpose, however, the layer determining the mechanical properties, such as the tension, pressure, torsion and bending strength is the layer or the layers of the film strip windings, and not the layer of optional further coatings.

In order to be able to carry out the production method in an economical manner, the theoretically endlessly long catheter longitudinal section may be produced and subsequently cut to the desired lengths, wherein the cut length sections are then subjected to further processing, such as welding to a balloon, or attaching grip pieces.

According to the invention a catheter is further provided, wherein the catheter longitudinal section is produced in that a film is provided in a strip shape, the film strip is wound in the manner of a screw such that the side edges of the film strip extending in the manner of a thread overlap the film strip, and a bonded connection is created in the overlapping area such that the longitudinal section has a closed and at least fluid-tight surface.

Advantageously, the catheter is such a catheter as is provided by means of the production of a longitudinal section according to the invention in accordance with at least one of the claimed production methods. The catheter receives inherent reinforcement from the winding of the film strips, which is comparable to the inherent reinforcement obtained by means of wire mesh or additional layers in conventional catheters. As opposed to conventional catheters improved intrinsic gliding properties, or also an improved grip, for example on the exterior surface of the catheter shaft, can further be realized by means of the influence of the transitions in the overlapping regions. The catheter or catheter shaft according to the invention may be utilized for rinsing vessels, preferably in organic tissue, and for attaching spreading elements, such as a balloon. Such a balloon is advantageously welded onto the distal end of the catheter shaft, in an at least fluid-tight, preferably in a gas-tight manner. Therefore, the catheter or the catheter shaft forms a fluid line for the inflation and deflation of the balloon attached thereto. Conditional of manufacturing, the catheter according to the invention has a low wall thickness enabling the realization of a relatively large inner diameter with a comparably low outer diameter. This means that the catheter according to the invention is easier to insert or guide through vessels, and simultaneously has only a slight blockage effect within the vessel due to the low cross-section thereof. Due to the improved intrinsic gliding properties a tool may be easily inserted and passed through the interior of the catheter or the catheter shaft. Due to the fact that the catheter shaft is merely wound and bonded, the production thereof is simple and uncomplicated such that the production can be realized in a cost-effective manner.

The catheter is advantageously embodied in that it has areas including different diameters at the shaft. For this purpose the conical shape is particularly favorable, wherein the end of the conical shape having the smaller diameter should preferably be the distal end of the catheter shaft. In case a conical shape or a hollow conical section shape is present, the windings of the film strips are therefore present as spatial spirals. The different diameter regions of the catheter shaft or of the longitudinal section thereof may be influenced by the incline of the coiling and the overlapping degree. The areas of the longitudinal sections of the catheter having a smaller diameter, therefore also comprise a lower axial resisting torque. The axial resisting torque, however, may in turn be increased in that more coils are present across a certain length, thus slightly increasing the wall thickness in this area. In a particularly advantageous manner the areas having the reduced diameter may be utilized for insertion into narrower vessels.

A particular embodiment of the catheter according to the invention is that at least a partial area of the catheter shaft has an elliptical hollow cross-section. Such an elliptical hollow cross-section can be created, for example, by means of winding the film strip onto an elliptical mandrel. Areas of the catheter shaft having an elliptical cross-section may be advantageously utilized for positioning in vessels, in which a wider and flatter embodiment of the catheter is of advantage.

In order to realize the small outer diameter and the small wall thickness of the catheter or the catheter shaft, the invention provides that the film strip thickness is smaller than 80 μM. In this manner catheters can be produced, which are substantially thinner than currently known. Therefore, catheters having diameters of 0.5-1 mm can be produced, which are particularly suited for PTCA or neurovascular applications.

In the production of a catheter being wound from at least two film strips, each of the film strips utilized may be comprised of a different material. It is of advantage that the film strip creating the interior wall has good gliding properties due to low roughness values, and that the film strip forming the outer surface has a good ability for welding, for example, onto a balloon. As already mentioned with regard to the production method, the invention may provide that a film strip comprised of two layers is provided, which is subsequently wound into a catheter shaft. For this purpose the two layers of the film strip to be wound may also be comprised of different materials. The different materials utilized may be substantially incompatible with each other with regard to the ability for welding thereof, such as polyamide and polyimide. However, in order to still be able to realize a bonded connection by means of welding of the film strips, polyamide-typical components may be added, for example, to the polyimide.

It is generally provided that the film strip is comprised of a biocompatible polymer. At least one of the film strips utilized should be weldable to a nylon balloon. Furthermore, it is of advantage to utilize film strips having fluorine plastic or a texture in order to realize good gliding properties on the inner surface of the catheter shaft.

Advantageously, the polymers utilized for the production of the film strip include PEEK, PI, PS, PES, and/or PA 12, which is particularly suitable for attaching conventional PA 12 balloons.

Advantageously, the longitudinal section of the catheter produced according to the invention has a hollow cylindrical shape or a hollow conical section shape. In order to produce a hollow conical section shape film strips having an inconsistent width may be utilized, which are optionally wound while changing the incline. In this manner a different bending stiffness can be realized in different sections.

Furthermore, cambers may be created in sections of the catheter shaft such that the catheter shaft has an increased axial resisting torque in these sections. Said areas improved with regard to the bending stiffness can be utilized particularly in those sections of the catheter shaft, in which an increased bending load is expected upon use.

According to the invention a device is provided for realizing the production method according to the invention, and for the production of the catheter or the longitudinal sections thereof, which comprises a unit for the torque-driven accommodation of a winding mandrel, and a unit for guiding a film strip onto the winding mandrel that rotates in the operating state thereof, wherein the device is embodied such that a relative movement can be carried out in a controlled manner between the winding mandrel and the guiding position of the film strip. This means that the device ensures that a winding mandrel rotates, and film strip is wound onto the winding mandrel in a guided manner, wherein the guide is embodied such that the same may, for example, perform a translator movement relative to the winding mandrel such that an incline of the film strip windings can be created.

For this purpose the accommodation of the winding mandrel may be integral part of the winding mandrel. In this embodiment the winding mandrel itself is torque-driven. If the accommodation is present as a separate component, the winding mandrel can be removed from the accommodation, thus resulting in the possibilities for guiding the wound film material into a cooling section and/or for improved handling during further processing.

Advantageously the device according to the invention comprises a unit for fixing at least one film strip onto the winding mandrel. This means that, for example, the winding mandrel may comprise a clamping or similar device, which serves for mounting a film strip end on or at the winding mandrel. In this manner only the film strip or the films strips need to be attached to the winding mandrel for the production of the film strip windings, and the same must be brought to rate, wherein the film strip is guided in a translator displaceable guide unit and wound onto the winding mandrel in order to obtain a catheter, or also a longitudinal section of the catheter according to the invention after subsequent bonded connection to the film strips or the overlapping thereof.

For this purpose the device may be embodied such that the film strip can be wound by means of applying tension. This means that after the film strip is attached to the winding mandrel, the same is subjected to tension by means of the film strip guide unit such that the same is elastically deformed to a certain degree, thereby applying radially acting pressing forces onto the winding mandrel, or onto the film strip present underneath the respective film strip to be wound, in the wound state on the winding mandrel due to the elastic reset force. Said radial pressing forces have an advantageous effect on the forming of the smooth, fluid-tight surface, and on performing the bonded connection by means of welding or gluing.

### DESCRIPTION OF THE DRAWINGS

The invention is described based on the attached drawings.

FIG. 1 is a longitudinal section of the catheter in a hollow cylindrical shape, produced according to the invention.

FIG. 2 is a longitudinal section of a catheter having a conically shaped area, produced according to the invention.

FIG. 3 is the conically shaped longitudinal section illustrated in FIG. 2, including an illustration of the winding courses of all film strips processed.

### DETAILED DESCRIPTION OF THE INVENTION

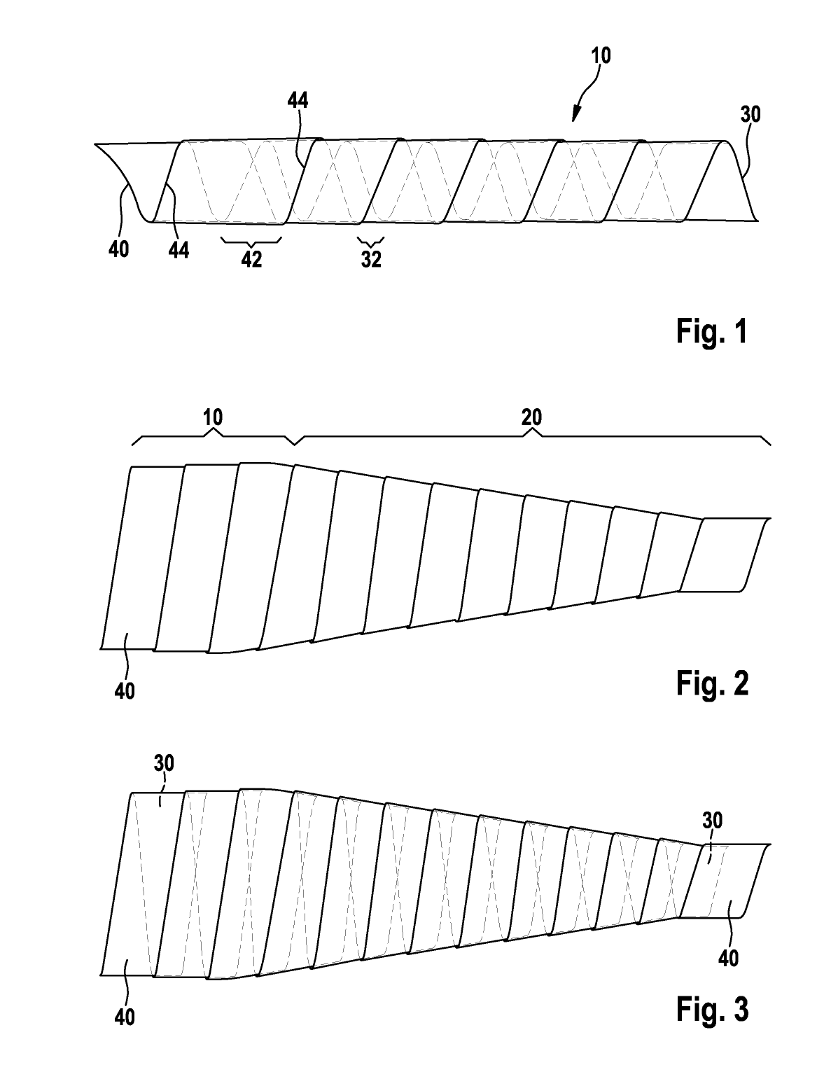

The longitudinal section of the catheter shaft illustrated in FIG. 1 is a hollow cylindrical area 10 having uncut ends. This means that the ends of the longitudinal section illustrated in FIG. 1 should be cut for an easier connection of a balloon or a grip by means of welding or gluing. The longitudinal section illustrated in FIG. 1 is wound from two film strips 30 and 40, wherein the second film strip 40 is wound over the windings of the first film strip 30. It can be seen that both film strips 30 and 40 have opposite inclines. This means that the first film strip 30 has a clockwise incline, and the second film strip 40 has a counter-clockwise incline. This results in the two film strips 30 and 40 overlapping each other in a crosswise manner. Such crosswise overlapping has the advantage of a uniform distribution of the overlapping regions across the lateral surface of the longitudinal section, or of the catheter shaft. Therefore, uniform torsion stiffness is ensured upon initiating a clockwise or also counter-clockwise torsion torque. For this purpose the first film strip 30 illustrated as a dotted line is wound such that the windings thereof at least partially overlap in an overlapping region 32. This means that with the welding of the overlapping regions 32 of the first film strip 30, or with the gluing of said overlapping regions an already functional catheter shaft could be produced. However, as illustrated in FIG. 1, the windings of the second film strip 40 are placed additionally across the windings of the first film strip 30 for the purpose of cross-sectional reinforcement, wherein said second film strip 40 also realizes overlapping regions 42 by means of its own windings. The bonded connection of the film strips can therefore comprise the connection of the first film strip 30 with itself in the overlapping regions 32 thereof, and also the bonded connection in the overlapping regions 42 of the second film strip 40. However, a longitudinal section of a catheter having substantially two tubes that are embodied independently of each other and are arranged inside of each other, which would theoretically be able to carry out relative movements toward each other, would be produced in this manner. In order to avoid this, and in order to increase the strength of the catheter shaft, the invention may therefore provide that the bonded connection is not only carried out in the overlapping regions 32 and 42 of the individual film strips among each other, but that the bonded connection also comprises the connection of the first film strip 30 to the second film strip 40.

The side edges 44 of the second film strip 40 illustrated in FIG. 1 extend parallel to each other such that a consistently wide overlapping region 42 is embodied at a constant incline of the winding.

Differing from the embodiment illustrated in FIG. 1 it is also possible that, as mentioned, the longitudinal section is provided solely by the windings of one film strip, or that in case of the use of multiple film strips the film strips wound on top of each other have the same direction of incline.

The longitudinal section illustrated in FIG. 1 can be created in that the film strips 30 and 40 a re wound onto a winding mandrel, which substantially has an outer diameter complementary to the inner diameter of the windings of the first film strip 30.

FIG. 2 show a particular embodiment of a catheter or catheter shaft according to the invention, which has a hollow cylindrical area 10 and a hollow conical section shaped area 20. This means that the catheter shaft has areas including different diameters in the hollow conical section shaped area 20.

As illustrated in FIG. 3, a longitudinal section extending in such a conical manner may also be realized by means of winding one film strip, or even two film strips 30 and 40. For this purpose the two film strips 30 and 40 are wound such that they cross each other in a similar manner as illustrated in FIG. 1. The winding of the film strips 30 and 40 onto a winding mandrel having a conical shape lends itself to realize the conical shape, or the film strip may be initially present in spiral shape, and is subsequently pulled apart into a three-dimensional conical spiral shape.

In case of a winding of the film strips onto a winding mandrel it should advantageously be provided that said winding mandrel is coated with Teflon or silicone oil, or similar separating means, before winding in order to prevent a welding or gluing of the film strips to the mandrel, thus ensuring a good removal after completed production of the longitudinal section of the catheter.

It will be apparent to those skilled in the art that numerous modifications and variations of the described examples and embodiments are possible in light of the above teaching. The disclosed examples and embodiments arc presented for purposes of illustration only. Therefore, it is the intent to cover all such modifications and alternate embodiments as may come within the true scope of this invention.

### List of Reference Symbols

10 Hollow cylindrical area

20 Hollow conical section shaped area

30 First film strip

32 Overlapping region of the first film strip

40 Second film strip

42 Overlapping region of the second film strip 42

44 Side edge

What is claimed is:

1. A method for the production of at least one longitudinal section of a catheter, wherein the method comprises the winding of film, characterized in that the film is provided in a strip shape, the film strip is wound in the manner of a screw such that the side edges of the film strip extending in the manner of a thread overlap the film strip, and a bonded connection is created in the overlapping region such that the longitudinal section has a closed and at least fluid-tight surface.

2. The method for the production of a longitudinal section of a catheter according to claim 1, characterized in that the bonded connection is realized by means of welding.

3. The method for the production of a longitudinal section of a catheter according to claim 2, characterized in that the welding is carried out while increasing the pressure on the film strip to be connected.

4. The method for the production of a longitudinal section of a catheter according to claim 1, characterized in that the bonded connection is realized by means of gluing.

5. The method for the production of a longitudinal section of a catheter according to claim 1, characterized in that only one film strip is wound for the production of the longitudinal section, wherein said film strip partially overlaps itself.

6. The method for the production of a longitudinal section of a catheter according to claim 1, characterized in that at least two film strips are wound for the production of the longitudinal section, wherein each of the film strips partially overlaps itself and/or at least one of the other film strips.

7. The method for the production of a longitudinal section of a catheter according to claim 1, characterized in that the film strip is produced by means of extrusion, and the side edges of the film strip provided, extending in longitudinal direction, are aligned in the extrusion direction.

8. The method for the production of a longitudinal section of a catheter according to claim 1, characterized in that the film strip is wound onto a winding mandrel.

9. The method for the production of a longitudinal section of a catheter according to claim 1, characterized in that the film strip is initially present as a strip spiral, and is subsequently pulled apart along the center axis of the spiral such that windings overlapping each other are created, the overlapping regions of which are subsequently bonded to each other.

10. A catheter, wherein at least one longitudinal section is produced at least partially by means of winding film, characterized in that the longitudinal section is produced according to the method according to claim 1.

11. The catheter according to claim 10, characterized in that the longitudinal section has areas comprising different diameters.

12. The catheter according to claim 10, characterized in that the film strip thickness is smaller than 80 μm.

13. The catheter according to claim 10, characterized in that the longitudinal section is wound from at least two film strips made of different material.

14. The catheter according to claim 10, characterized in that the longitudinal section has a hollow cylindrical shape, or a hollow conical section shape.

15. A device for carrying out the method according to claim 1, characterized in that the device comprises the following:
a unit for the torque-driven accommodation of a winding mandrel, and a unit for guiding film strip onto the winding mandrel that rotates during the operating state thereof, wherein the device is embodied such that a relative movement can be carried out in a controlled manner between the winding mandrel and the guiding position of the film strip.
